(19) United States
(12) Patent Application Publication
(10) Pub. No.: US 2018/0042651 A1
(43) Pub. Date: Feb. 15, 2018

(54) A BONE ROD

(71) Applicant: The Sydney Children's Hospitals Network (Randwick and Westmead), New South Wales (AU)

(72) Inventors: David Graham Little, New South Wales (AU); Mathemorn Lan, New South Wales (AU); Justin Bobyn, New South Wales (AU)

(73) Assignee: The Sydney Children's Hospitals Network (Randwick and Westmead), New South Wales (AU)

(21) Appl. No.: 15/550,924

(22) PCT Filed: Feb. 15, 2016
(86) PCT No.: PCT/AU2016/050097

§ 371 Date: Aug. 14, 2017

(30) Foreign Application Priority Data
Feb. 17, 2015 (AU) 2015900530
Nov. 13, 2015 (AU) 2015904687

(51) Int. Cl. A61B 17/72; A61B 17/70

(52) U.S. Cl. CPC A61B 17/7216; A61B 17/7014; A61B 17/7241; A61B 17/7283; A61B 2017/681

(57) ABSTRACT

A device and method for correcting a rotational deformity, for use in lengthening a bone or aiding in stabilising a fractured bone. When used to correct a rotational deformity, the device comprises a first member and a second member moveably connected to each other such that when the two members are moved apart along a first axis, the first and/or second members rotate relative to each other about the first axis.

### CROSS-REFERENCE TO RELATED APPLICATIONS

The present application claims priority from Australian Provisional Patent Application No 2015900530 filed on 17 Feb. 2015 and Australian Provisional Patent Application No 2015904687 filed on 13 Nov. 2015, the contents of which are incorporated herein by reference.

### TECHNICAL FIELD

The present disclosure relates to orthopaedic devices and methods for correction of deformities in bones, including deformities in bones at or adjacent a growth plate.

### BACKGROUND

A growth plate, also known as the epiphyseal plate or physis, is a growing area of tissue adjacent to the diaphysis at each end of a long bone. The growth plate determines the future length and shape of the mature bone. The plate is found in growing children and adolescents. When growth is complete, the growth plate closes and is replaced by an epiphyseal line of solid bone.

In some children, a growth plate will grow non-uniformly, with growth on one side of the growth plate being faster than on another side, causing an angular or a rotational deformity of the bone. Angular and rotational deformities can also be congenital, caused by trauma or result from bone diseases. Neuromuscular disorders including cerebral palsy can cause rotational and angular malalignment via abnormal muscle forces acting on the bone. Rotational malalignment can also be idiopathic, that is, of no known cause.

With rotational deformities, the most common current method of treatment involves subjecting the patient to an invasive osteotomy, whereby a region of the bone is cut and rotated to achieve re-alignment, typically about a joint, although in effect any section of the bone can be cut and rotated to achieve correction.

Rotational guided growth has been attempted by the use of non-orthogonal tension band plates (Arami Al, Bar-On E, Herman A, Velkes S, Heller S. Guiding femoral rotational growth in an animal model. J Bone Joint Surg Am. 2013 Nov. 20; 95(22):2022-7). This method causes a torsional moment on the growth plate and effects a rotational deformity of the bones as the plates move from a non-orthogonal to an orthogonal position. However, once the plates are orthogonally oriented, growth (rotational or longitudinal) can no longer occur, limiting the benefits of this method. Furthermore, with this method, calculating how many degrees of inter plate angle (IPA) is required to correct a rotation is not straightforward, and depends on the length of the plate as well as the growth rate. As such this method has not been widely adopted.

In some cases, growth of one bone in a limb may be faster than that of the corresponding bone on the other limb resulting in a biomechanical imbalance. Distraction osteogenesis is a method that is commonly used to lengthen the shorter limb. Following an osteotomy, an external fixator is used to distract the bone segments at a rate that will allow subsequent bone formation. This technique requires the use of an external fixator for up to a year, during which time osteopenia may occur due to stress shielding by the external fixator and non-use of the bone. This bone catabolism may result in up to 60% of the bone mineral content of the limb being lost, the resultant morbidity including prolonged healing time, re-fracture following frame removal and delays in rehabilitation due to most patients requiring a plaster cast post-lengthening. A device and method which avoids the prolonged use of an external fixator is desirable in leg lengthening.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

According to one aspect, the present disclosure provides an orthopaedic device for securing between first and second regions of bone separated by a growth plate, the orthopaedic device comprising:

a first member having an end to secure to the first bone region; and

a second member having an end to secure to the second bone region;

wherein the first and second members are moveably connected to each other such that when the ends are moved apart along a first axis, the first and/or second members rotate relative to each other about the first axis.

The first member may extend from a first end to a second end, with the first end secured to the first bone region. Further the first member may include a first bone fixation region. The first bone fixation region may be located at or adjacent to the first end of the first member.

The second member typically also extends from a first end to a second end, wherein the first end is secured to the second bone portion. The second member may also include a second bone fixation region. The second bone fixation region may be located at or adjacent to the first end of the second member. The second fixation region may be similar, or the same as, the first bone fixation region. Alternatively, the second bone fixation region may differ from the first bone fixation region.

The first fixation region may comprise one or more holes through the first member. Similarly, the second fixation region may comprise one or more holes through the second member. In this example, the holes are configured to receive a locking member such as a locking pin, screw, bolt, blade, fin or K-wire to secure said first and/or second member to surrounding bone. An inner surface defining the hole of the first and/or second member may be at least partially or fully threaded. Alternatively the inner surface is substantially smooth. Where threaded, the hole may receive a threaded locking member.

The inner surface of the first and/or second members may define a substantially cylindrical hole through the first and/or second members. The holes may comprise a number of cross-sectional shapes including circular, oval, rectangular, square or other. Further, the holes may include one or more outwardly extending channels. In one embodiment, the holes comprise a central hole and two opposed outer recesses which extend from the central hole. Alternatively, the hole includes more than two outer recesses, extending from the central hole.

In another embodiment, the first fixation region may comprise a threaded length of the first end of the first member. Similarly, the second fixation region may comprise a threaded length of the first end of the second member. In such embodiments, the first end of the first member and the first end of the second member may be screwed into the surrounding bone to secure the first and second members respectively thereto.

The first and second members are typically linearly slidable along at least said first axis relative to each other. Such linear sliding of the first and second members relative to each other is typically driven by the growth of a bone across the growth plate, referred to as “guided growth”.

The configuration of the first and/or the second member and the connection therebetween, is such that linear movement driven by the growth of the bone effects a rotational movement of the first and second members relative to each other about the first axis.

In one embodiment, the first member includes an outer surface comprising one or more rotational feature. The one or more rotational feature of the first member may be complementary in size and configuration to one or more rotational feature of the second member. The relationship between the one or more rotational feature of the first member and the one or more rotational feature of the second member is such that a rotational movement is caused around the first axis upon an axial movement along the first axis.

In one embodiment, the one or more rotational feature may comprise ridges and/or channels on an outer surface of one of the first or second member and complementary crests and/or grooves on an inner surface of the other member such that one member may be moveable within the other member to effect a rotational movement around the first axis.

The one or more rotational feature of the first member may include one or more ridges along at least a length of the outer surface of the first member. Additionally or alternatively, the rotational features of the first member may include one or more channels along at least a length of the outer surface of the first member. The one or more ridges and/or one or more channels may extend helically along a length of the outer surface of the first member.

In one embodiment, the one or more channels and/or ridges may extend helically around a full circumferential turn of the first member. Further, the one or more channels and/or ridges may extend such that they make more than one circumferential turn around the first member. Alternatively, the one or more channels and/or ridges may only partially extend around the circumference of the first member such as to make less than one turn of the first member. In one embodiment, the one or more channels and/or ridges extend around less than 75% of the circumference of the first member. Further, the one or more channels and/or ridges may extend around 70%, 60%, 50%, 40%, 30%, 20%, 10% or less of the circumference of the first member.

In one embodiment, the one or more rotational feature comprises a single ridge extending helically along a length of an outer surface of the first member. Alternatively, or additionally, the one or more rotational feature may comprise a single channel extending helically along a length of an outer surface of the first member.

Alternatively, the rotational feature may comprise two helically extending channels and two helically extending ridges. Further, the first member may comprise three helically extending channels and three helically extending ridges. In other embodiments, the first member may comprise 4, 5, 6, 7, 8, 9, 10 or more helically extending channels defining respective helically extending ridges therebetween.

In a further embodiment, the entire outer circumference of at least a length of the first member may be a ridged configuration, that is, with multiple helically oriented channels and ridges.

The one or more channels and/or ridges of the first member may be elongate and extend along a majority of the length of said first member. Alternatively, the one or more channels and/or ridges do not extend along the full length of the first member and in particular do not extend to the first fixation region which is at, or adjacent to, the first end.

The first member may comprise a first mating region which extends from a junction with the first fixation region towards the second end of the first member. The one or more rotational feature may be located on an outer surface of the first mating region.

The one or more channels and/or ridges of the first member may extend from a second end of the first member towards the first end of the first member. In one embodiment, the one or more channels and/or ridges extend along 90% of the length of the first member. In another embodiment, the one or more channels and/or ridges may only extend along around 20% or less of the length of the first member. In another embodiment, the one or more channels and/or ridges may extend along 30%, 40%, 50%, 60%, 70%, or 80% of the length of the first member.

The one or more elongate channels and/or ridges may extend towards the first end of the first member in either a clockwise or counter-clockwise direction around the outer surface of the first member, depending upon the desired direction of rotation of the first member.

The first member may be substantially straight along its length. Alternatively the first member may comprise one or more angled portions. In one embodiment, a region of the first member adjacent said second end may be angled relative to the remainder of the first member. Alternatively, or additionally, a region of the first member adjacent the first end may be angled relative to the remainder of the first member.

The first member may be substantially solid along a majority of its length. Alternatively, the first member may be at least partially tubular. One or both of the first end and the second end of the first member may be open. At least the first end of the first member may be bevelled to avoid damaging the intra-osseous tissue of a patient during insertion through the medullary canal of a bone.

The one or more rotational feature of the first member may extend from a junction with the bone fixation region of the first member and towards the second end of the first member and form the mating region of the first member. Typically, the rotational features extend to the second end. A junction between the bone fixation region and the mating region may be substantially stepped with a shoulder formed on the bone fixation region. The shoulder may act as a stop to prevent full insertion of the first member into the second member. In this embodiment, the shoulder of the bone fixation region is configured to abut with the second end of the second member to prevent further insertion of the first member into the second member as discussed in more detail below.

The first member may have a number of cross sectional shapes including circular, oval, square or rectangular. In one embodiment, the cross section of at least the mating region of the first member is square. The mating region in this embodiment comprises a substantially helically twisted elongate body having four helically oriented elongate surfaces, each elongate surface extending from the junction with the first bone fixation region towards the second end of the first member. Each elongate surface may comprise said rotational feature described above.

The elongate surfaces of the first member may extend along a majority of the length of the mating region. Alternatively, the elongate surfaces of the first member may extend along a partial length of the mating region.

The elongate surfaces may helically extend towards the second end of the first member in either a clockwise or counter-clockwise direction around the outer surface of the first member, depending upon the desired direction of rotation of the first member. Two adjacent helically orientated elongate surfaces may define a helical edge therebetween. The elongate surfaces may rotationally mate with a complementary surface of the second member to cause a rotational movement around the first axis upon axial movement along said first axis.

The elongate surfaces may be substantially planar or, alternatively, may present a curved or rounded surface.

The second member may be at least partially tubular and may include an internal lumen defined by an internal wall. The internal lumen may be sized to receive at least part of the first member.

The internal wall of the second member may comprise one or more rotational features which are complementary to the rotational features of the first member.

The second member may be cylindrical and have a substantially uniform diameter along its length. Alternatively, the diameter and/or cross sectional shape of the second member may vary along its length. The second member may have any cross-sectional shape including circle, oval, square or rectangular. The second member is typically of a complementary shape relative to the first member to receive the first member therein.

The one or more rotational feature of the second member may include one or more crests along at least a length of the internal wall of the second member. Additionally or alternatively, the one or more rotational feature of the second member may include one or more crests along at least a length of the internal wall of the second member. The one or more crests and/or one or more grooves may extend helically along a length of the internal wall of the second member.

In one embodiment, the one or more crests and/or grooves may extend helically around a full circumferential turn of the internal wall of the second member. Further, the one or more crests and/or grooves may extend such that they make more than one circumferential turn around the internal wall of the second member. Alternatively, the one or more crests and/or grooves may only partially extend around the internal wall of the second member such as to make less than one turn of the internal wall. In one embodiment, the one or more crests and/or grooves extend around less than 75% of the circumference of the internal wall of the second member. The one or more crests and/or grooves may extend around 70%, 60%, 50%, 40%, 30%, 20%, 10% or less of the circumference of the internal wall of the second member.

The one or more rotational feature of the second member may comprise a single ridge on the internal wall of the second member. Alternatively, or additionally, the rotational feature may comprise a single groove in the internal wall of the second member. The single ridge and/or groove may extend helically along a length of the internal wall of the second member.

The second member may comprise two grooves. Alternatively, the second member may comprise 3 grooves. In other embodiments, the second member may comprise 4, 5, 6, 7, 8, 9, 10 or more grooves.

The number of elongate grooves and crests of the second member may be dependent upon the number of ridges and channels of the first member. Typically, the grooves of the second member are sized to receive the ridges of the first member and the channels of the first member sized to receive the crests of the second member.

The first and second members may be brought into engagement such that the ridges of the first member are slidably moveable along the grooves of the second member and the crests of the second member are slidably moveable in the channels of the first member.

In a further embodiment, the internal wall may be helically, multiply ridged around its entire circumference to match a similarly ridged structure of the outer surface of the first member.

The grooves and/or the crests of the second member may extend along a majority of the length of said first member. The length of the grooves and the crests is typically determined by the length of the second member and in particular the length of the channels and ridges of the second member.

In the embodiment described above wherein the first member comprises a square cross sectional mating region having helically arranged elongate surfaces, the internal wall of the second member may comprise complementary helically oriented guiding surfaces to mate with said elongate surfaces of the first member. The guiding surfaces of the second member may extend from the second end and towards the first end of the second member. The guiding surfaces may extend the full length of the internal wall of the second member. Alternatively, the guiding surfaces extend only partially along the length of the internal wall. In one embodiment, a length of the internal wall adjacent to the second end of the second member comprises the guiding surfaces with the remainder of the internal wall defining a lumen having a substantially circular cross section. The guiding surfaces may guide the elongate surfaces of the first member in a desired orientation when the first and second members are in a rotational engagement.

In another embodiment, the second member comprises one or more guiding nodules which extend from the internal wall. Typically the guiding nodules may extend from the internal wall adjacent the second end of the second member although one or more nodules may be positioned anywhere along the length of the internal wall of the second member. The guiding nodules may be received within the channels of the first member. Due to the helical orientation of the channels, such engagement between the nodules and the channels effects a rotational movement of the first member relative to the second member upon linear movement of said first member relative to the second member.

The grooves and crests or guiding surfaces of the second member may extend in the same orientation, that is, clockwise or counter-clockwise, relative to the channels and ridges or the elongate surfaces of the first member.

The degree of helical extension of the channels and/or ridges or elongate surfaces of the first member and the grooves and/or crests or the guiding surfaces of the second member may depend on the desired degree of rotation, around the first axis, of the first and second members relative to each other. For example, in one embodiment, the degree of rotation of the first member relative to the second member is no greater than 90°. In another embodiment, the degree of rotation of the first member relative to the second member may be 80, 70, 60, 50, 40, 35°, 30°, 25°, 20°, 15°, 10°, 5° or less. In a further embodiment, the degree of rotation of the first member relative to the second member is no greater than 30°. Where the degree of rotation of the first member relative to the second member is less than 5°, the degree of rotation may be 4°, 3°, 2°, 1° or 0°.

The device described above may be used to correct both a rotational deformity in addition to use in lengthening a bone. In this embodiment, it is envisaged that an osteotomy would be made in the bone of the patient away from the growth plate and the device positioned to allow the growth of the bone to both rotate the bone and lengthen the bone across the osteotomy.

The second member may be longer in length than the first member. In this embodiment, the crests and/or grooves or the guiding surfaces may extend from the second end of the second member towards the first end. The elongate crests and/or grooves or the guiding surfaces may extend along the entire length of the second member. Alternatively, the elongate crests and/or grooves or guiding surfaces extend less than 90%, 80%, 70%, 60%, 50%, 40%, 30%, 20% of the length of the second member.

The second fixation region of the second member, at or adjacent to the first end, may be devoid of the elongate grooves and crests or guiding surfaces. In another embodiment, the first and second members may be substantially the same length. In a further embodiment, the second member is shorter in length than the first member.

The second member may be substantially straight along its length. Alternatively the second member may comprise one or more angled portions. One or more angled portions may be particularly desirable if the second member is introduced into the bone in a retrograde manner.

In one embodiment, the second member comprises an angled primary portion, an intermediate portion and a terminal portion. The primary portion may extend from the first end. The intermediate portion extends from a junction with the primary portion. The terminal portion extends from a junction with the intermediate portion and towards the second end of the second member.

The terminal portion of the second member may extend along the first axis. The intermediate portion may extend along a second, different axis to the first axis. The second axis may be angled relative to the first axis. In one embodiment, the second axis is angled relative to the first axis from between 1° to 15°. Alternatively the second axis is at an angle from between 3° and 8° and preferably around 4°, 5°, 6° or 7° relative to the first axis. In one embodiment, the angle is 5°.

The angulation of the terminal portion relative to the intermediate portion is preferably such that when the first member is in engagement with the second member, the second member is positioned substantially orthogonal to a growth plate. Such positioning of the second member may allow an optimal rotational moment around the growth plate when implanted in a bone of a subject.

The primary portion of the second member may extend along a third axis, different to the first and/or second axes. The third axis typically extends at an angle of between 1° and 15° to the second axis. Preferably the angle is between 5° and 12°; more typically between 8° and 10° relative to the second axis. In one embodiment, the primary portion extends along the third axis at 10° relative to the second axis of the intermediate portion. This angle is designed to allow for optimal entry to the bone and in particular for trochanteric entry into the bone of a subject. Once implanted in, for example, a femur, the primary portion may sit within the greater trochanter.

The intermediate member may be substantially straight or alternatively, the intermediate member may comprise a bow between the junction with the primary portion and the junction with the terminal portion. The intermediate portion may, when implanted in a bone, bow anteriorly to posteriorly. Alternatively, when implanted, the intermediate portion may bow posteriorly to anteriorly; laterally to medially or medially to laterally.

The first and/or second ends of the second member may be bevelled to avoid damaging the intra-osseous tissue of a patient during insertion of the second member through the medullary canal of the bone.

In use, the two members may be mated together by inserting the first member into the second member. The first member may be at least partially inserted into the second member. Typically, the second end of the first member is aligned with the second end of the second member such that the ridges of the first member align with the grooves in the internal wall of the second member and/or the crests of the second member aligned with the channels of the first member. The second member may then be inserted into the second member and rotated relative to the second member to a desired positioning for insertion into the intramedullary canal of a bone.

The first member may be inserted into the second member to an extent that at least a length of the first member adjacent to the first end extends from the second end of the second member. As noted above, a shoulder formed between the bone fixation region and the mating region of the first member may abut with the second end of the second member to prevent further insertion into the second member.

Preferably, in an implantation configuration, at least the first fixation region extends beyond the second end of the second member to allow a user to lock the first fixation region to the first bone region.

When referring to the devices of the disclosure when implanted or being implanted into or onto a bone of a subject, the terms “proximal” and “distal” will be used. It is to be understood that the term “proximal” means nearest the point of origin or attachment of an anatomical structure and the term “distal” means situated away from the point of origin or attachment of an anatomical structure. For example, the proximal end of the femur is closest to the hip whereas the distal end of the femur is closer to the knee of a subject. Similarly, the proximal end of the radius is closest to the shoulder and the distal end of the humerus is closer to the elbow.

The first end of the first member may be secured at the first fixation region to the first bone region which may be distal relative to the growth plate of the bone. The first end of the second member may be secured at the second fixation region to the second bone region which may be proximal to the growth plate.

With the bone fixation regions of the first and second members fixed to respective, opposed, bone regions across the growth plate, growth of the bone across the growth plate causes a linear movement along the first axis and the first member linearly moves along the first axis relative to the second member.

In an embodiment, as the first member moves linearly relative to the second, fixed member, it is also forced into a rotational movement relative to second member.

Because the first member is also fixed to the bone at the first bone region, rotation of the first member relative to the second member will cause a corresponding rotation of the first bone region, driven by the linear growth of the bone across the growth plate. The first bone region may rotate either clockwise or counter-clockwise relative to the second bone region depending upon the orientation of the rotational features of both the first and second members.

The first bone region may comprise a region of bone distal to the growth plate. However, the first bone region may, in some instances, be located proximal to a growth plate. For example, if the device is implanted in a proximal region of a tibia, the first member may be secured proximally relative to the growth plate.

The second member may rotate in a counter direction to the first member. However, because the second member is relatively fixed in the second bone region, such counter rotation is relatively small compared to the rotation of the first member and the first bone region. Thus, linear growth across the growth plate predominantly exerts a rotational force upon the first bone region to induce rotation of the first bone region in a desired orientation.

In another aspect, the present disclosure provides a method of correcting a rotational deformity in a bone including:

preparing an access region of the bone to insert a device along a length of an intramedullary canal of a bone, said device comprising a first member and a second member, said first and second members moveably connected to each other along a first axis and rotatably moveable about the first axis;

securing an end of the first member to a first bone region

securing an end of the second member to a second bone region;

wherein said first and second bone regions are separated by a growth plate; and wherein further, upon growth of the bone, the first and/or second members are caused to move apart along the first axis and rotate relative to each other about the first axis to cause a rotation of at least part of the bone.

The bone may be any bone of a subject. For example, the bone may be the femur, the tibia, the fibula, the humerus, the ulna, the radius, the clavicle or a vertebral bone.

In one embodiment, the bone comprises the femur. In this embodiment, the device may be used to correct femoral anteversion and antetorsion.

In correcting femoral anteversion and antetorsion, the first member may be fixed to the distal femoral epiphysis and the second member to femur anywhere proximal to the growth plate.

Preferably, in correcting anteversion and antetorsion of a bone, at least the distal femoral epiphysis is externally rotated relative to the proximal femur by the device of the present disclosure.

The orientation of the helical channels and ridges of the first member and/or the helical crests and grooves of the second member may determine the direction of rotation of the second bone region.

For example, in an embodiment wherein the device is implanted into a left femur of a patient to correct anteversion or antetorsion, the helical channels and ridges of the elongate surfaces of the first member and/or the helical crests and grooves or the guiding surfaces of the second member extend proximally to distally in a counter-clockwise direction. In the right femur, the helical channels and ridges or elongate surfaces of the first member and/or the helical crests and grooves or the guiding surfaces of the second member may extend proximally to distally in a clockwise direction.

In another embodiment wherein a bone exhibits retrotorsion or retroversion, the helical channels and ridges or the elongate surfaces of the first member and/or the helical crests and grooves or the guiding surfaces of the second member may be the reverse to the orientation required for anteversion and antetorsion of a bone. For example when implanted in a left femur of a patient, the helical channels and ridges or elongate surfaces of the first member and/or the helical crests and grooves or the guiding surfaces of the second member extend proximally to distally in a clockwise direction to effect internal rotation of the bone.

For a right femur exhibiting retrotorsion or retroversion, the helical and channels and ridges or elongate surfaces of the first member and/or the helical crests and grooves or the guiding surfaces of the second member may extend proximally to distally in an anti-clockwise direction to effect internal rotation of the bone.

The device of the present disclosure may also be used to rotate at least a first bone region of a tibia. In this embodiment, the first bone region may comprise the proximal epiphysis of the tibia.

To externally rotate the left tibia, the device may be implanted such that the helical channels and ridges or elongate surfaces of the first member and/or the helical crests and grooves or guiding surfaces of the second member extend proximally to distally in an anti-clockwise direction. Such a procedure may be employed to correct tibial torsion.

To externally rotate the right tibia, the device may be implanted such that the helical channels and ridges or elongate surfaces of the first member and/or the helical crests and grooves or guiding surfaces of the second member may extend proximally to distally in a clockwise direction.

To correct external tibial torsion the helical channels and ridges or elongate surfaces of the first member and/or the helical crests and grooves or guiding surfaces of the second member may be reversed in their orientation. For example, in the left tibia, the helical channels and ridges or elongate surfaces of the first member and/or the helical crests and grooves or guiding surfaces of the second member may extend proximally to distally in a clockwise direction.

To correct external tibial torsion of the right tibia, the helical channels and ridges or elongate surfaces of the first member and/or the helical crests and grooves or guiding surfaces of the second member may extend proximally to distally in an anti-clockwise direction.

To rotate a left humerus, the device may be implanted such that the helical channels and ridges or elongate surfaces of the first member and/or the helical crests and grooves or guiding surfaces of the second member extend proximally to distally in an anti-clockwise direction to externally rotate the humerus. Internal rotation may be achieved by the reverse of this, that is, the helical channels and ridges or elongate surfaces of the first member and/or the helical crests and grooves or guiding surfaces of the second member extend proximally to distally in a clockwise direction.

To rotate a right humerus, the device may be implanted such that the helical channels and ridges or elongate surfaces of the first member and/or the helical crests and grooves or guiding surfaces of the second member extend proximally to distally in a clockwise direction to externally rotate the humerus. Internal rotation may be achieved by the reverse of this, that is, the helical channels and ridges or elongate surfaces of the first member and/or the helical crests and grooves or guiding surfaces of the second member extend proximally to distally in an anti-clockwise direction.

In a further aspect, there is provided a spinal device for securing between first and second regions of vertebral bone, the spinal device comprising:

a first elongate member having a first connection end to secure to the first bone region; and

a second elongate member having a second connection end to secure to the second bone region;

wherein the first and second members are moveably connected to each other such that when the ends are moved apart along a first axis, the first and/or second members rotate relative to each other about the first axis.

The first member may be attached to a first vertebra and the second member attached to a second vertebra, spaced from the first vertebra such that the spinal device spans one or more vertebral disc spaces.

The first and second members of the spinal device may include any one of the features of the first and second member described in relation to the first aspect.

The spinal device may span any region of the vertebral column including vertebrae of the cervical, thoracic and lumbar regions. Therefore, the lengths of each elongate members may vary depending upon where they are positioned in the body and in particular how many vertebrae the two members are required to span.

One of the first and second members may be connected at its connection end to any one of the vertebrae of the spinal column while the other member is connected at its connection end to a spaced vertebra. While the connection ends of the first and second end may be on adjacent vertebrae, it is envisaged that the spinal device spans a plurality of vertebrae. For example, the second or the first member may be secured to a T1 vertebra and the other member attached at its connection end to a T2, T3, T4, T5, T6, T7 or T8 vertebrae.

In one embodiment a plurality of spinal devices may be used in a rotation correct procedure. The devices may be aligned in series along a length of the vertebral column. Alternatively, or additionally, the devices may be aligned in a substantially parallel arrangement relative to each other, typically connected medially or laterally relative to a spinous process of a vertebra(e). The device(s) may be secured to posterior elements of the vertebrae or secured anteriorly to the vertebral bodies of the respective vertebrae.

Spinal deformities may develop in a child as they grow, with at least parts of the vertebral column rotating undesirably as the child grows. Scoliosis is an example of a deformity associated with the rotation of several vertebrae. While most commonly affecting the thoracic region, scoliosis may also cause a curvature of the cervical and lumbar regions. In some instance, the lumbar region curves to compensate for an initial curvature of the thoracic vertebrae which may result is an S-shaped curve of the vertebral column. As part of a fusion correction of a scoliotic spine, the affected part of the vertebral column is de-rotated as part of the procedure prior to fusion and fixation.

The spinal device herein described may be used to treat a rotation deformity including but not limited to scoliosis.

Typically, the first and the second members are attached to respective vertebral bones by one or more fixation members. Examples of suitable fixation members include a pedicle screws, laminar hooks or sublaminar wires.

The first and second members are typically linearly slidable along at least said first axis relative to each other. Such linear sliding of the first and second members relative to each other is typically driven by the growth of the subject and the vertebra(e) across the growth plate(s). The “motor” to drive the linear movement of the two members relative to each other is often referred to as “guided growth”. It should be appreciated that the spinal device may include a further motor as described further below.

The configuration of the first and/or the second member and the connection therebetween, is such that linear movement driven by the growth of the vertebrae over a selected part of the vertebral column effects a rotational movement of the first and second members relative to each other about the first axis. Depending upon the positioning of the spinal device in the patient, the rotational movement of the first and second members may cause a rotation of the vertebrae spanned by the ends of the spinal device. Rotation of the vertebrae in this manner may provide a counter rotation to the pathological rotation such as would occur in the process of, for example, scoliosis.

Particularly, when the first and second members are fixed to spaced respective vertebral bone regions, growth of the vertebral bones across the growth plates of the vertebrae in which the members are fixed and the vertebra(e) in between causes a linear movement along the first axis and the first member linearly moves along the first axis relative to the second member. As the first member moves linearly relative to the second member, it is also forced into a rotational movement relative to the second member.

Because the first member is also fixed to the bone at the first bone region, rotation of the first member relative to the second member will cause a similar rotation of the first bone region, driven by the linear growth of the bone across the growth plates. This rotation may be translated to each of the connecting vertebrae between the two connection ends of the device to cause a rotation of the vertebrae across which the device spans.

The first bone region may rotate either clockwise or counter-clockwise relative to the second bone region depending upon the orientation of the rotational features of both the first and second members.

The spinal device may be connected to the vertebrae of a subject by a number of means including, but not limited to pedicle screws, laminar hooks or sublaminar wires. When secured anteriorly, the device may be connected to the bone by vertebral screw bodies.

The devices of the present disclosure may be introduced into a bone using either a retrograde or an antegrade procedure.

When in position, preferably the first member rotates relative to the second member at between 5° and 30° per approximately 10 mm of growth. Alternatively, the growth may be between 10° and 20°; more typically between 10° and 15° per approximately 10 mm of growth.

While it is envisaged that growth of the bone is a sufficient driver of rotation of the first and second member relative to each other, in one embodiment, the device may include a separate “motor”. Examples of the type of “motor” include but are not limited to a manual expansion; a simple mechanical device such as a spring or clockwork device; an osmotic pump device; a programmable electric motor; a motor powered by external ultrasound energy; a magnetic motor. In one embodiment, the motor may be an external fixator, including a monolateral or ring device.

Rate of growth when using a motor may be optimised to avoid damage of the growth plate and to substantially mimic the known rate of growth of the bone. In another embodiment, the rate may be slightly faster than the known growth rate. In one embodiment the rate may be not more than double the rate of natural growth of the bone.

The device for correcting a rotational deformity may also be used to stabilise a fracture. Typically, the device is positioned across a fracture rather than across a growth plate to hold the fracture fragments together and allow the fracture to heal. However, if the device is also positioned across both a fracture and a growth plate, it is envisaged that it may be used to both stabilise a fracture and also allow correction of a rotational deformity. In this example, the device for correcting rotational deformity is configured such that there is zero degrees of rotation between the first and second elongate members.

In one aspect there is provided an orthopaedic device for lengthening a bone of a subject, the orthopaedic device comprising a first elongate member and a second elongate member arranged along a first axis:

the first elongate member extending from a first end to a second end, the first end configured to secure to a first bone region and at least part of the first elongate member adjacent to the second end comprising a lumen defined by an internal wall of the first member;

the second elongate member extending from a first end to a second end, the second end configured to secure to a second bone region and at least a length of the second elongate member adjacent to the first end is receivable within the lumen of the first elongate member;

the internal wall of the first elongate member comprises one or more linear guide features engageable with complementary linear guide features of the second elongate member; wherein

when the one or more linear guide members of the first and second elongate members are engaged with each other and the first end of the first member and the second end of the second elongate member are moved apart from each other, the first and/or second elongate members move substantially linearly relative to each other along the first axis.

The linear guide elements of the device of the above aspect which is used for lengthening a bone may comprise the previously described ridges, channels, grooves and crests but it is to be understood that, rather than helically arranged, said guide elements are substantially linearly arranged and, for example, extend along an axis which is substantially parallel to the first axis of the device.

The device for lengthening a bone according to the above aspect may include a motor to drive the movement of the first and second elongate members relative to each other. The motor may include a simple mechanical device such as a spring or clockwork motor; an osmotic pump device; a programmable electric motor; a motor powered by external ultrasound energy; or a magnetic motor.

The device for lengthening a bone may also be used to stabilise a fracture. Typically, because lengthening is not required in this example, the device when used to stabilise a fracture does not include a motor and is instead positionable across a fracture to hold the fracture fragments together and allow the fracture to heal.

A further aspect provides a method of correcting a length deformity in a bone including:

making a cut across the bone to form a first bone region and a separated second bone region;

preparing an access region of the bone to allow insertion of a guidewire through an intramedullary canal of the bone and across the cut;

inserting an orthopaedic device over the guidewire such that the orthopaedic device bridges the cut in the bone, the orthopaedic device comprising
a first elongate member and a second elongate member arranged along a first axis; the first elongate member extending from a first end to a second end, the first end configured to secure to the first bone region and wherein at least part of the first elongate member adjacent to the second end comprises a lumen defined by an internal wall of the first elongate member; the second elongate member extending from a first end to a second end, the second end configured to secure to the second bone region and wherein at least a length of the second elongate member adjacent to its first end is received within the lumen of the first elongate member; wherein the internal wall of the first elongate member comprises one or more linear guide features which engage with complementary linear guide features of the second elongate member

securing the first end of the first elongate member to the first bone region

securing the second end of the second elongate member to the second bone region;

causing the first and/or second elongate members to move apart from each other such that the first and/or second elongate members move substantially linearly relative to each other along the first axis.

The first and/or second elongate members may move apart at a controlled rate such that the first and second bone regions are distracted, that is, they are kept in a spaced relationship from one another to promote bone growth at the cut in the bone and thus lengthen the bone. The rate of movement may be controlled by a motor such as a spring or clockwork motor; an osmotic pump device; a programmable electric motor; a motor powered by external ultrasound energy; or a magnetic motor.

The first and second members of the present disclosure as described in each aspect above may be made from a number of biocompatible materials including a metal or a metal alloy. Alternatively, the first and second members may be made from a polymeric material. Examples of suitable materials include stainless steel and its alloys, titanium and its alloys, cobalt chrome and its alloys, tantalum and its alloys, polyether ether ketone (PEEK), MP35N and its alloys, graphite/pyrocarbon.

In another example, the bone fixation region may comprise a roughened, etched, porous or ribbed surface for bone and tissue ingrowth and to promote bone fixation for subsequent development of mechanical fixation. The bone fixation region may include a coating or be impregnated with an agent to promote bone fixation. The surface of the bone fixation region may include a Hydroxyapatite (HA) coating. In this regard, the surface may be a beaded porous surface, wire mesh porous surface, selective sintered porous surface or other trabecularized metal scaffold.

Similarly, the locking screws, wires, bolts or fins may comprise a roughened, etched, porous or ribbed surface for bone and tissue ingrowth and to promote bone fixation for subsequent development of mechanical fixation. In one embodiment, the surface of the locking screws, wires, bolts or fins may include a Hydroxyapatite (HA) coating. In this regard, the surface may be a beaded porous surface, wire mesh porous surface, selective sintered porous surface or other trabecularized metal scaffold. Achieving bony ingrowth and subsequent mechanical fixation reduces the chance of loosening of the locking screws, wires, bolts or fins, relative to the fixation region and backing-out of the screw. Loosening of the implant relative to the bone may reduce the rotational force experienced by the bone as it grows due to tension not being maintained between the implant and the bone.

The outer surfaces of the first and second members may be coated with a drug which may be eluted over time. In this regard, one or more of the outer surfaces may be coated for the elution of any one or more of the following: antibiotics, antimicrobials, an osteoinductive agent (including but not limited to an osteogenic protein, or a growth factor, or a member or the TGF-beta superfamily). Preferably the osteoinductive agent is an osteogenic protein. Preferably the osteogenic protein is a bone morphogenetic protein (BMP), preferably recombinant human form selected from rhBMP-1, rhBMP-2, rhBMP-3, rhBMP-4, rhBMP-5, rhBMP-6, rhBMP-7, rhBMP-8a, rhBMP-8b, rhBMP-9, rhBMP-10, and rhBMP-15. More preferably the BMP is rhBMP-2 or rhBMP-7.

In one embodiment, the osteogenic protein is rhBMP-2. In other preferred embodiments, suitable osteogenic proteins include rhBMP-7 (OP-1) currently approved for clinical use. rhBMP-4, rhBMP-6, and rhBMP-9 are other preferred embodiments.

In another embodiment, the eluted agent acts via the Wnt pathway. Agents such as antibodies to sclerostin, Dkk1 and Dkk2, SFRP1 and SFRP2 are envisaged. Antibodies that augment the Wnt pathway via LRP 4 5 or 6 could also be eluted. In one embodiment, small molecule drugs such as GSK3 antagonists such as lithium and its salts and AR28(AZD9828) and related compounds may also be eluted to upregulate Wnt pathway activity.

In other embodiments the osteoinductive agent may be a growth factor such as platelets/platelet derived growth factor (PDGF), insulin-like growth factor (IGF), vascular endothelial growth factor (VEGF), and/or a member of the TGF-beta superfamily such as TGF-beta 1, TGF-beta 2, TGF-beta 3, growth and differentiation factors (GDFs), fibroblast growth factors, activins, inhibins, or other specific activators of these pathways. The active agent may also comprise additional agents such as the Hedgehog, Frazzled, Chordin, Noggin, Cerberus and Follistatin proteins, or small molecule, protein, or antibody-based agents that antagonize Dickkhopf-1, Sclerostin, or other member of the Wnt signalling pathway. The active agent may also include antibodies, peptides, or soluble receptors affecting signal transduction via these pathways (e.g. tyrosine kinase growth factor receptors, insulin receptors, activin-like kinase receptors, bone morphogenetic protein receptors, fibroblast growth factor receptors, and transforming growth factor receptors) in full length, truncated, or with point mutations. In a preferred embodiment the factor that is antagonized is Myostatin (GDF-8).

In other embodiments, agents known to effect the delivery and presentation of growth factors to cells are included or added. Such agents include heparin sulphate and other glycosaminoglycans and their components, as well as specific binding proteins such as TGF-β binding protein.

In another embodiment the eluted agent is an anti-resorptive agent. Preferred anti-resorptive agents include bisphosphonates such as zoledronic acid, pamidronic acid, ibandronic acid, etidronic acid, alendronic acid, risedronic acid, or tilurondic acid as well as other non-specified bisphosphonates or their salts. Other anti-resorptive agents include IKK inhibitors (such as PS-1145), Osteoprotegerin (OPG), inhibitors of Cathepsin K, Chloride Ion Channel Blockers, Proton pump inhibitors, and antagonists of RANKL (Denosumab), and others.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

FIG. 1a is a lateral view of a device of the disclosure in an implantation configuration;

FIG. 1b is a lateral view of the device of FIG. 1a with parts of the device spaced and rotated relative to each other;

FIG. 2a is an anterior-posterior view of the device of FIG. 1a;

FIG. 2b is an anterior posterior view of the device of FIG. 1a with parts of the device spaced and rotated relative to each other;

FIG. 3a is a perspective view of a second member of the device of FIG. 1a;

FIG. 3b is a close up view of an end of one embodiment of a second member;

FIG. 3c is a close up view of an end of the embodiment of the second member shown in FIG. 3a;

FIG. 4 is a perspective view of a first member of the device of FIG. 1a;

FIG. 5a is a perspective view of the device of FIG. 1a when implanted in a left femur of a subject;

FIG. 5b is a perspective view of the device of FIG. 1a after the left femur has grown relative to the femur shown in FIG. 5a;

FIG. 6a is a perspective view of the device of a further embodiment when implanted in a left femur of a subject;

FIG. 6b is a perspective view of the device of FIG. 6a after the left femur has grown relative to the femur shown in FIG. 5a;

FIGS. 7a and 7b are perspective views of a second member of the device of FIG. 6a;

FIG. 8a, is a lateral view of a first member of the device of FIG. 6a;

FIG. 8b is an anterior-posterior view of the first member of the device of FIG. 6a;

FIG. 8c is a perspective view of the first member of the device of FIG. 6a;

FIGS. 9a, 9b and 9c are perspective views of another embodiment of the second member of the disclosure;

FIGS. 10a, 10b and 10c are perspective views of another embodiment of the first member of the disclosure;

FIG. 10d is a perspective view of the first member of another example of the device of the disclosure;

FIG. 11a is a side view of one embodiment of a locking mechanism;

FIG. 11b is a perspective view of the locking mechanism of FIG. 9a;

FIG. 12a is a side view of a further embodiment of a locking mechanism;

FIG. 12b is a perspective view of the locking mechanism of FIG. 10a;

FIG. 13 shows further examples of the device of the present disclosure when used to correct abnormally rotated spine;

FIG. 14 shows longitudinal radiographic images of the two experimental animals of Example 1 shown at the time of implantation (Surgery) and at the final harvest (3 months);

FIG. 15 shows X-ray of the implanted devices of Experiment 1 ex-vivo at the three month harvest; and

FIG. 16 shows the rotation of the devices of Experiment 1 in the femora, post-harvest as measured by the angle of pins inserted into part of the device.

### DESCRIPTION OF EXAMPLES

In one example, an orthopaedic device 1 includes a first member 10 and a second member 50. Second member 50 is configured to receive first member 10. The assembly of first member 10 and second member 50 forming device 1 may be implanted into a bone of a patient. Particularly, the device 1 is implanted through the medullary canal of a bone.

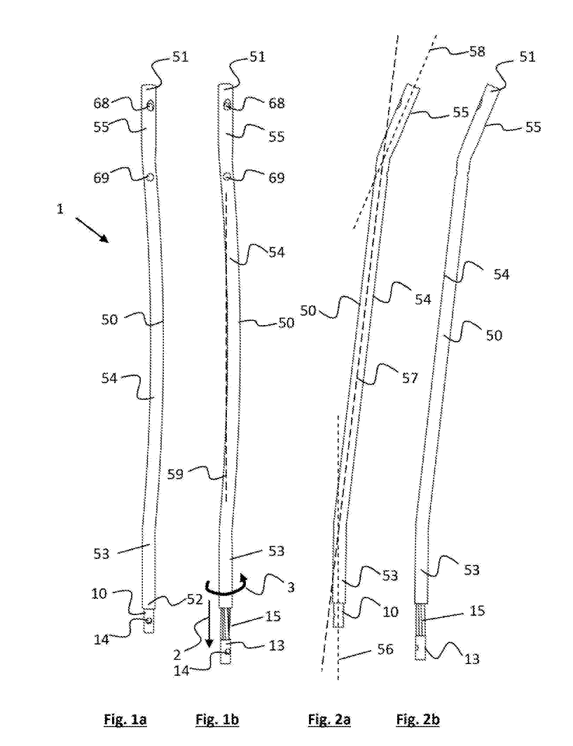

The device 1 is positioned over a growth plate of a bone and the engagement between the first member 10 and the second member 50 is such that they are axially and rotatably moveable relative to each other as the bone grows. FIGS. 1b and 2b depict an arrangement of first member 10 and second member 50 relative to each other after the bone, in which device 1 is implanted, has grown. First member 10 moves axially and distally from second member 50 in a direction shown by arrow 2. As the first member 10 moves axially in the direction shown by arrow 2, it also rotates relative to second member 50 as shown by arrow 3.

First member 10 extends from a first end 11 to a second end 12. A first fixation region 13 is located adjacent to first end 11. In use, the first member 10 is fixed to the bone at the first fixation region 13. The first fixation region 13 comprises a substantially solid cylindrical length of the first member 10 adjacent first end 11. A hole 14 is formed in the first fixation region 13 to receive a locking member 191 to secure at least the fixation region 13 to the surrounding bone. While only one hole 14 is depicted, in other examples, the first fixation region 13 may comprise multiple holes.

First member 10 further includes a mating region 15 which extends from the first fixation region 13 to the second end 12. The mating region 15 is configured to mate with a complementary structure of the second member 50. In one example, mating region 15 comprises ridges 20a, 20b and 20 c. Channels 21a, 21b and 21c are formed between these ridges. Ridges 20a, 20b and 20c extend from a junction with the first fixation region 13 in a helical configuration around an outer wall of the mating region 15.

A shoulder 16 is formed at the junction of the first fixation region 13 and the mating region 15 in the depicted example. Shoulder 16 abuts with end 52 of second member 50 when device 1 is in its implantation configuration. Shoulder 16 may act as a stop and prevent the first fixation region 13 sliding into the second member 50. This allows hole 14 to remain exposed to permit fixing of the first member 10 to the surrounding bone.

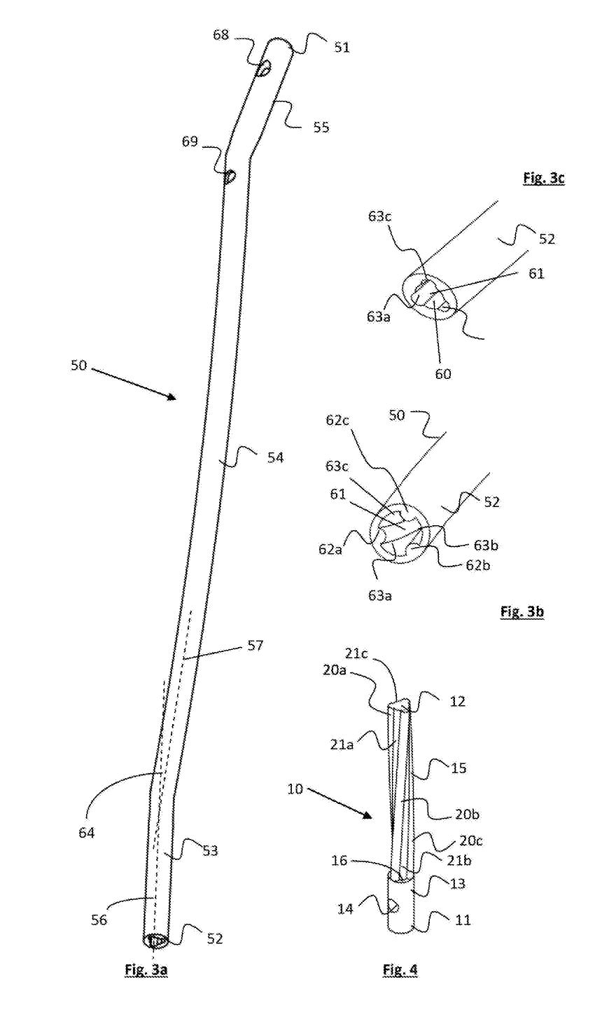

In the example shown in FIG. 3a, second member 50 comprises an elongate body which extends from a first end 51 to a second end 52. At least a region of the second member 50 adjacent to the second end 52 is tubular to receive the first member 10 as shown in more detail in FIGS. 3b and 3c. An internal wall 60 defines a lumen 61 to receive part of the first member 10. In the example shown in FIG. 3b, the internal wall 60 comprises crests 62a, 62b and 62c. Between crests 62a, 62b and 62c are grooves 63a, 63b and 63c. The crest and grooves extend from the second end 52 of second member 50 in a helical configuration as can best be seen by crest 62a in FIG. 3b.

In the example shown in FIG. 3c, the internal wall 60 does not define the same protruding ridges 62a, 62b or 62c and instead includes relatively deeper grooves 63a, 63b and 63c to receive the ridges of the first member 10.

The helical configuration and the dimensions of crests 62a, 62b and 62c and/or grooves 63a, 63b and 63c of second member 50 complement the helical arrangement of first member 10 such that the ridges 20a, 20b and 20c of the first member are moveable along respective grooves 63a, 63b and 63c and similarly, crests 62a, 62b and 62c are moveable along respective channels 21a, 21b and 21c.

To achieve an implantation configuration as depicted in FIG. 1a, first member 11 is inserted into the lumen 61 of second end 52 of second member 50. Particularly, mating region 15 is inserted into second member 50 until shoulder 16 abuts with end 52 such that only the first fixation region 13 extends from the second end 52 of second member 50. In the implantation configuration, hole 14 in the first fixation region 13 is exposed to receive a locking member and thus allow fixing of at least the first fixation region 13 to the surrounding bone. To insert the first member 10 into second member 50 requires a rotation of the two members relative to each other to guide ridges 20a, 20b and 20c along respective grooves 63a, 63b and 63c and similarly, crests 62a, 62b and 62c along respective channels 21a, 21b and 21c.

The second member 50 comprises a terminal portion 53, an intermediate portion 54 and a primary portion 55. FIG. 2A, depicts an anterior posterior view of device 1 showing terminal portion 53 extending along a first axis 56. The intermediate portion 54 extends along a second, different axis 57. The primary portion 55 extends along an axis 58 which is different to both the first axis 56 and second axis 57.

As can be seen in the lateral views of FIGS. 1a and 1b, intermediate portion 54 may not be straight and may bow along its length, either anteriorly or posteriorly. The bowing may be seen relative to line 59 drawn from one end of the intermediate portion 54 to the other. Such bowing of the second member in this manner may be of particular relevance if the device is implanted in an antegrade fashion into a femur of a subject, the femur having a natural anterior bow along at least part of its length.

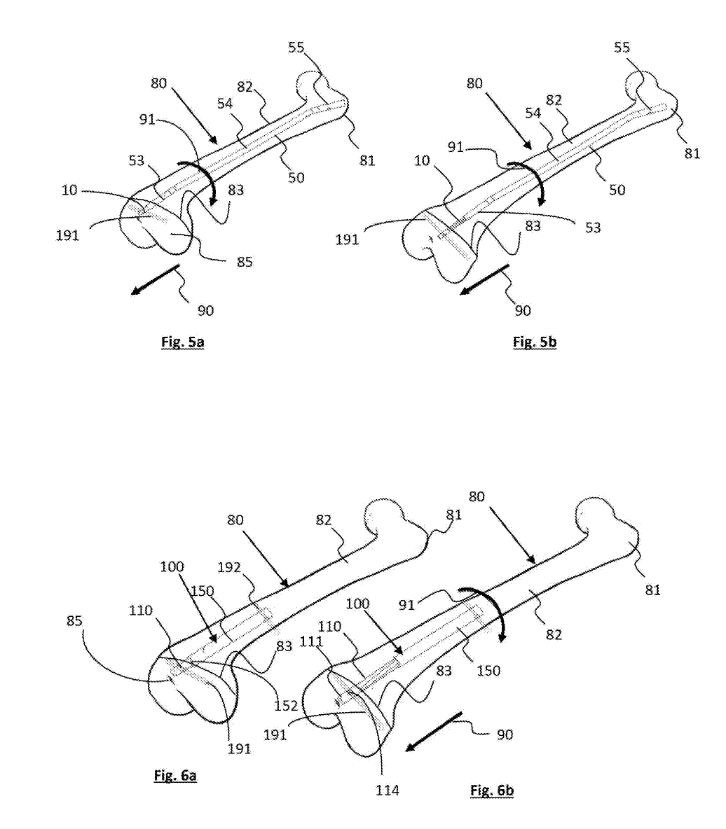

The angles between the different portions 53, 54 and 55 may depend upon the bone in which device 1 is implanted. In the examples shown in FIGS. 1a, 1b, 2a, 2b and 3a, the second member 50 is insertable into the medullary canal of a femur 80 in an antegrade manner, that is via the greater trochanter 81, to a final position as shown in FIGS. 5a and 5b. Typically the angle 64 formed between axis 56 and axis 57 is around 5° to 6°.

Primary portion 55 sits within the greater trochanter 81 of the femur 80 when implanted as shown in FIGS. 5a and 5b. Intermediate portion 54 extends through the diaphysis 82 and terminal portion 53 extends from the diaphysis 82 towards distal growth plate 83. Second end 52 of second member 50 is shown just proximal to the growth plate 83, and in practice, it is desirable to avoid the second member 50 extending across the growth plate. Because the second member 50 may have a larger diameter than the first member 10 it may cause relatively more damage than if only first member 10 extends over the growth plate.

Second member 50 may also comprise a hole 68 adjacent first end 51 to receive a locking member 192 to secure at least the primary portion 55 of second member 50 to surrounding bone. A further hole 69 is shown in the intermediate portion 54 which may receive another locking member and therefore further secure the second member 50 to surrounding bone. The intermediate portion may comprise further holes to further secure the first member 50 in the bone.

For a rotational correction of the left femur, the device 1 is assembled into the implantation configuration shown in FIG. 1a and inserted, first member 10 first, through an entry point in the greater trochanter 81. The assembly of first member 10 and second member 50 is moved through the medullary canal until it is positioned as shown in FIG. 5a. First member 10 is secured to bone distal the growth plate 83 in the distal epiphysis 85 by inserting a locking member 191 through hole 14. Although not depicted in FIGS. 5a and 5b, a similar locking member is inserted through one or both of holes 68 and/or 69 to secure the second member 50 to surrounding bone in the greater trochanter and/or the diaphysis 82. Additional locking holes may also be incorporated anywhere along intermediate portion 54 to provide additional fixation.

In this example, the deformity to be corrected is an internal rotation of the femur 80 with correction requiring an external rotation of the femur 80 in the direction shown by arrow 91.

The first member 10 is positioned substantially orthogonal with the growth plate 83 to drive the correct rotation of the femur 80.

With the device in situ and first member 10 secured across the growth plate 83, axial growth of the femur 80 as depicted by arrow 90 exerts an axial force on first member 10 relative to second member 50 to axially pull first member 10 from second member 50 and in the direction of the growth of the femur 80. Due to the helical engagement between ridges 20a, 20b and 20c and grooves 63a, 63b and 63c and similarly, crests 62a, 62b and 62c with respective channels 21a, 21b and 21c, first member 10 is forced into a relative rotational movement in the direction of arrow 90 as the femur 80 grows. In turn, because the first member 10 is fixed to bone in the distal epiphysis 85, a rotational force is exerted by the first member 10 to at least the distal epiphysis to externally rotate a distal region of the femur 80 in the direction of arrow 91.

Therefore, the motor to drive the rotational force to the device 1 and the femur 80 is growth of the femur 80 itself. In other examples, correction may also be aided by an artificial motor.

Once the femur 80 has been optimally rotationally corrected as shown in FIG. 5b, the device 1 may be surgically removed. Alternatively, the device 1 may be designed such that the first member 10 becomes disengaged from the second member 50 after a pre-determined axial and rotational movement. In another example, the grooves 63a, 63b and 63c and crests 62a, 62b and 62c do not extend to end 52 such that there is a non-helically threaded region or a gap adjacent the distal end 52 of second member 50. Upon certain lengthening of the femur 80 and thus rotational movement of both the first member 10 relative to second member 50, second end 12 of first member 10 passes into the non-helically threaded region adjacent end 52 and thus the ridges 20a, 20b and 20c and channels 21a, 21b and 21c of first member terminate their rotational engagement with respective crests 62a, 62b and 62c and grooves 63a, 63b and 63c. While further growth of femur 80 will cause an axial movement of first member 10 relative to second member 50 in the direction of arrow 90, the relative rotational movement is terminated.

Alternatively one or more locking members such as locking member 191 may be removed to terminate the rotational force on the bone.

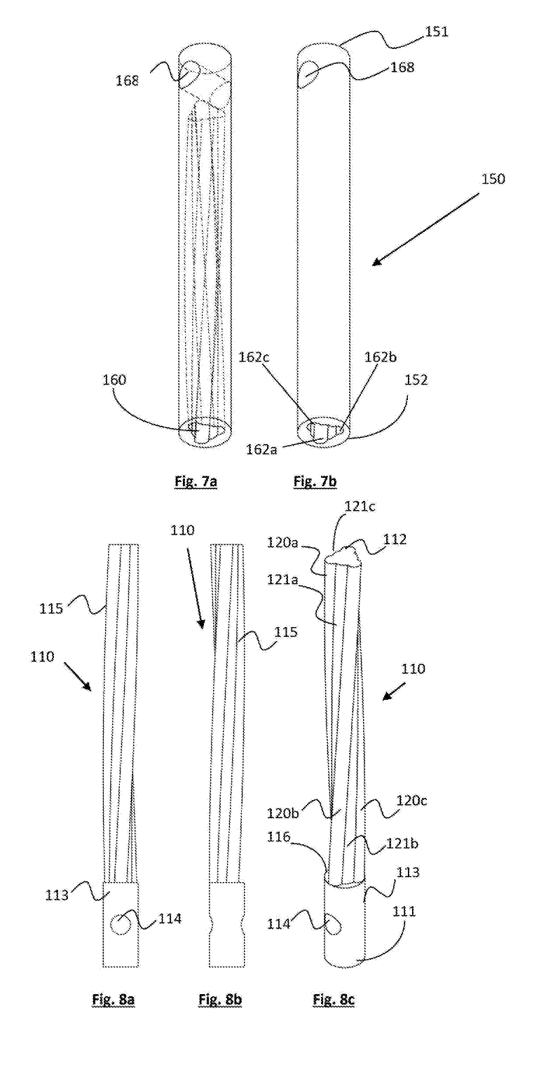

An example of a retrograde device 100 for retrograde insertion into a bone is depicted in FIGS. 7a and 7b, 8a to 8c and shown by way of example in a femur in FIGS. 6a and 6b. The retrograde device 100 is implantable in the femur 80 and across growth plate 83 via an entry point in the distal epiphysis 85 of femur 80 rather than entering via the greater trochanter 81 as depicted in FIGS. 5a and 5b.

Notably, while implanted in a retrograde manner for the femur 80, the depicted device 100 is suitable for antegrade implantation in a tibia.

First member 110 extends from a first end 111 to a second end 112. A first fixation region 113 is located adjacent first end 111. In use, the first member 110 is fixed to the bone at the first fixation region 113. The first fixation region 113 is a substantially solid cylindrical length of the first member 110 adjacent first end 111. First fixation member 113 comprises a hole 114 to receive a locking member 191 to secure at least the fixation region 113 to the surrounding bone as shown in FIGS. 6a and 6b. Although not depicted, the first fixation region 113 may comprise multiple holes.

First member 110 further includes a mating region 115 which extends from the first fixation region 113 to the second end 112. The mating region 115 is configured to mate with a complementary structure of the second member 150.

Mating region 115 comprises ridges 120a, 120b and 120c. Channels 121a, 121b and 121c are formed between these ridges. Ridges 120a, 120b and 120c extend from a junction with the first fixation region 113 in a helical configuration.

A shoulder 116 is formed at the junction of the first fixation region 113 and the mating region 115. Shoulder 116 abuts with end 152 of second member 150 when device 1 is in its implantation configuration as shown in FIG. 6a.

Second member 150 comprises a substantially straight tubular body which extends from a first end 151 to a second end 152. An internal wall 160 defines a lumen 161 to receive part of the first member 110. The internal wall 160 comprises helically arranged grooves 163a, 163b and 163c. Because the second member is implanted in a retrograde fashion it is shorter in length than second member 50 which is designed for antegrade implantation.

The dimensions and helical configuration of grooves 163a, 163b and 163c are complementary to the helical arrangement of ridges 120a, 120b and 120c of first member 110. Specifically, ridges 120a, 120b and 120c are sized to be moveable along respective grooves 163a, 163b and 163c.

Second member 150 also comprises a hole 168 adjacent first end 151. Hole 168 is configured to receive a locking member to secure the first member 150 to surrounding bone.

To achieve an implantation configuration as depicted in FIG. 6a, first member 110 is inserted into the lumen 161 of second end 152 of the second member 150. Mating region 115 is inserted into second member 150 until shoulder 116 abuts with end 152 such that only the first bone fixation region 113 extends from the second end 152 of second member 150. In the implantation configuration, hole 114 in the first fixation portion 113 is exposed to receive a locking member and thus allow fixing of at least the first fixation region 113 to the surrounding bone. To insert the first member 110 into second member 150 requires a rotation of the two members relative to each other to guide ridges 120a, 120b and 210c along respective grooves 163a, 163b and 163c.

For a rotational correction of the left femur 80 as shown in FIGS. 6a and 6b, device 100 is assembled into the implantation configuration and inserted, second member 150 first, through an entry point in the distal epiphysis 85 and through the medullary canal until the first end 151 of second member 150 is positioned in the diaphysis 82 and the second end 152 of second member is positioned just proximal to the growth plate 83. First member 110 extends distally from end 152 of second member 150 and crosses the growth plate 83. First end 111 of first member 110 is positioned distal to the growth plate 83 and in the distal epiphysis of the femur 80.

First member 110 is secured to bone in the distal epiphysis 85 by inserting a locking screw 191 through hole 114. Similarly, locking member 192 is inserted through hole 168 of second member 150 to secure the second member 150 in the bone of the diaphysis 82.

In the example shown in FIGS. 6a and 6b, the device 100 is used to externally rotate the femur 80 to correct a deformity. In this example, the femur 80 of FIG. 6a is abnormally internally rotated and requires external rotation in the direction of arrow 91 for correction.

The implantation of device 100 orientates the first member 110 substantially orthogonal with the growth plate 83 to drive the correct rotation of the femur 80.

When the device 100 is implanted, first member 110 is secured across the growth plate 83. Growth of the femur 80 as depicted by arrow 90 exerts an axial force on first member 110 relative to second member 150 to distally pull first member 110 from second member 150 and in the direction of the growth of the femur 80. Due to the helical engagement between ridges 120a, 120b and 120c and grooves 163a, 163b and 163c, first member 110 is forced into a relatively rotational movement in the direction of arrow 91 as the femur 80 grows. In turn, because the first member 110 is fixed to bone in the distal epiphysis 85, a rotational force is exerted by the first member 110 to at least the distal epiphysis 85 to externally rotate a distal region of the femur in the direction of arrow 91.

Again, in this example, the motor to drive the rotational force of the device 100 and the femur 80 is growth of the femur 80 itself. In other examples, correction may also be aided by an artificial motor.

Once the femur 80 has been optimally rotationally corrected as shown in FIG. 6b, the device may be surgically removed. Alternatively, the device 100 may be designed such that the first member 110 become disengaged from the second member 150 after a pre-determined axial and rotational movement. In another example, the grooves 163a, 163b do not extend fully along the entire length of second member 150 Particularly, in an example there may be a gap between the second end 152 and the start of the grooves 163a, 163b and 163c such as to terminate rotational engagement of the first member 110 relative to the second member 150 at a certain point of lengthening of the femur 80, thus also terminating a rotational force on the femur 80.

Alternatively, one or more locking member(s) 191 may be removed to terminate the rotational force on the femur 80.

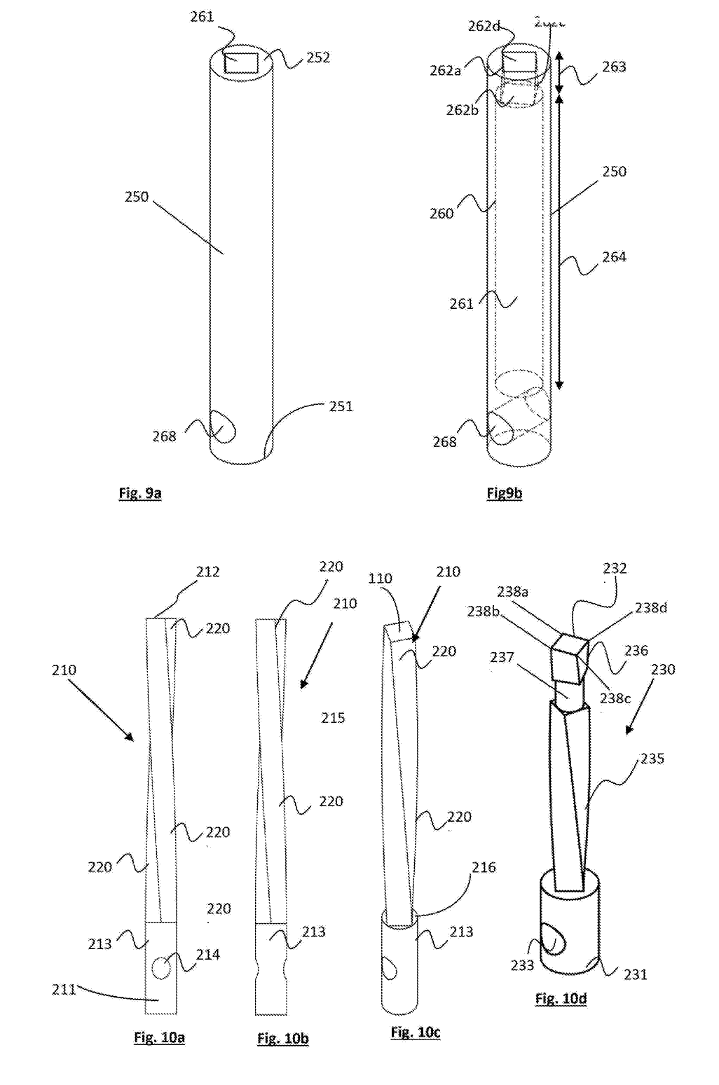

A further example of first member 210 is shown in FIGS. 10a, 10b and 10c. First member 210 comprises a substantially solid elongate body which extends from a first end 211 to a second end 212. First member 210 comprises a first fixation region 213 from which a mating region 215 extends towards second end 212. The first fixation region 213 is substantially cylindrical having a circular cross section. The first fixation region 213 comprises a hole 214 therethrough, the hole 214 sized to receive a locking member to fix the first member to surrounding bone.

The mating region 215 extends from a junction with first fixation region 213. A shoulder 216 is formed at the junction between first fixation region 213 and mating region 215, the shoulder acting as described above to prevent the entire length of the first member 210 moving into the second member 250.

Mating region 215 is depicted as having a square cross section although it is envisaged that any non-circular cross section would be a suitable adaptation of this example. The mating region comprises an elongate twisted body having four elongate surfaces 220a, 220b, 220c and 220d. Each elongate surface extends helically along the length of the mating region 215. FIG. 10a depicts a lateral view of the first member 210 wherein elongate surfaces 220a and 220b are visible. FIG. 10b depicts the first member 210 rotated approximately 90° to also show surfaces 220c and 220d.

FIG. 10d depicts a further example of first member 230. First member 230 also comprises a substantially solid elongate body which extends from a first end 231 to a second end 232. First member 230 comprises a first fixation region 233 from which a mating region 235 extends towards second ends 232. The first member 230 includes a head 236 which is positioned at second end 210. In the depicted example, the head 236 comprises a substantially cubed member although other shapes are envisaged. In the depicted example, the head 236 is spaced from the mating region 235 by spacer portion 237. When first member 230 is inserted into second member 250 ready for implantation in a subject, the head 236 prevents the first member 230 from readily disengaging from second member 250 because the configuration of the head 236 is such that it is offset relative to squared lumen 261 of second member 250 such that its corners 238a, 238b, 238c and 238d abut with a respective guiding surfaces 262a, 262b, 262c and 262d.

The first fixation region 233 is substantially cylindrical having a circular cross section. The first fixation region 213 comprises a hole 214 therethrough, the hole 214 sized to receive a locking member to fix the first member to surrounding bone.

The mating region 215 extends from a junction with first fixation region 213. A shoulder 216 is formed at the junction between first fixation region 213 and mating region 215, the shoulder acting as described above to prevent the entire length of the first member 210 moving into the second member 250.

Mating region 215 is depicted as having a square cross section although it is envisaged that any non-circular cross section would be a suitable adaptation of this example. The mating region comprises an elongate twisted body having four elongate surfaces 220a, 220b, 220c and 220d. Each elongate surface extends helically along the length of the mating region 215. FIG. 10a depicts a lateral view of the first member 210 wherein elongate surfaces 220a and 220b are visible. FIG. 10b depicts the first member 210 rotated approximately 90° to also show surfaces 220c and 220d.

FIGS. 9a and 9b depict second member 250 which comprises a substantially straight tubular body extending from a first end 251 to a second end 252. Second member 250 may be used for both retrograde or antegrade implantation. If used for antegrade implantation, second member 250 may be substantially longer than depicted and generally configured as shown in FIG. 1a.

An internal wall 260 defines a lumen 261 to receive part of the first member 210. Although the entire length of internal wall 260 may include a complementary surface to engage with elongate surfaces 220a, 220b, 220c and 220d , in the example depicted in FIG. 9b, only a length 263 of internal wall 260 adjacent second end 252 comprises helically arranged complementary guiding surfaces 262a, 262b, 262c and 262d. The remainder length 264 of internal wall 260 is substantially circular in cross section and devoid of a rotational feature.

The dimensions and helical configuration of guiding surfaces 262a, 262b, 262c and 262d are complementary to the elongate surfaces 220a, 220b, 220c and 220d of first member 210. Specifically, elongate surfaces 220a, 220b, 220c and 220d are sized and configured to be moveable along guiding surfaces.

Second member 250 also comprises a hole 268 adjacent first end 251. Hole 268 is configured to receive a locking member to secure the first member 250 to surrounding bone.

To bring the first member 210 and second member 250 together, first member 210 is inserted into the lumen 261 of second end 252 of the second member 250. Mating region 215 is inserted into second member 250 until shoulder 216 abuts with end 252.

In an implantation configuration, mating region 215 extends into length 264 of the lumen 261 of second member 250 and part of the length of elongate surfaces 220a, 220b, 220c and 220d of first member 210 abut with guiding surfaces 262a, 262b, 262c and 262d of second member 250. The orientation of the elongate surfaces and corresponding guiding surfaces is such that it causes rotation of the first member 210 relative to the second member 250 when the first member 210 moves axially relative to the second member 250.

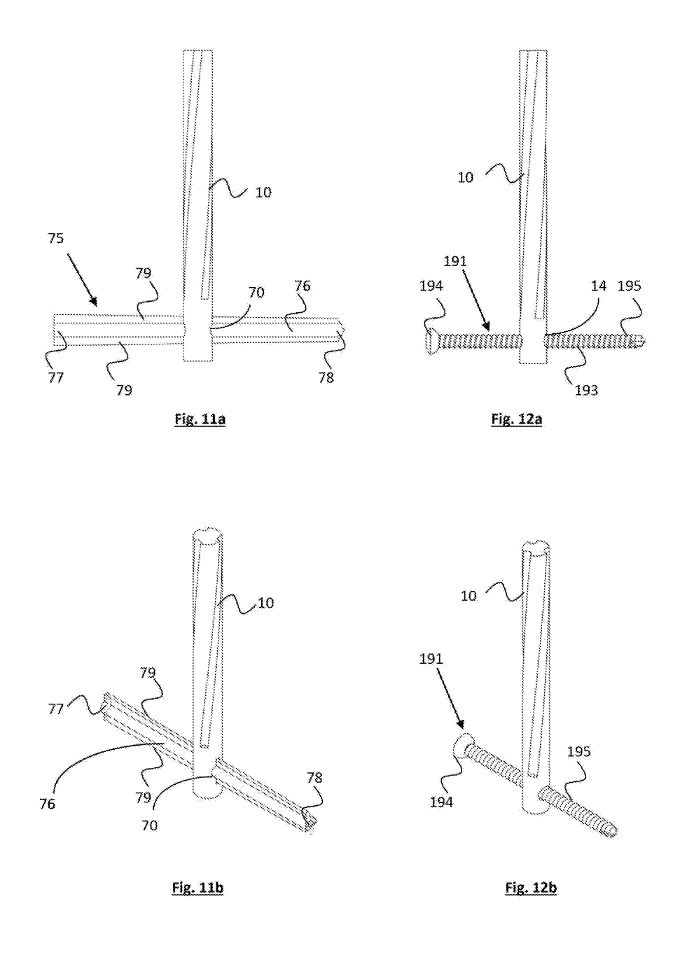

FIGS. 11 and 12 depict examples of locking mechanisms to secure either or both the first member 10, 110, 210 or second member 50, 150, 250 in surrounding bone. References in these drawings will be made to the features of device 1 but are equally applicable to the other examples depicted.

The first member 10 shown in FIGS. 11a and 11b comprises a key-shaped hole 70 which receives pin 75. Pin 75 comprises a tapered body 76 extending from a first end 77 to a second end 78. Fins 79 extend from body 76 and are received in complementary recesses of key-shaped hole 70. The fins 79 are configured to bite into surrounding bone as the tapered body 76 is wedged into the bone.

The first member 10 of FIGS. 12a and 12b comprises a substantially cylindrical hole 14 to receive a locking member 191. Locking member 191 in this depicted example is a locking screw 193 comprising a head 194 and an elongate threaded body 195 extending therefrom. The locking screw 193 may be screwed through hole 14 in either first member 10 or second member 50 such that the thread bites into surrounding bone to secure the first 10 or second member 50 to the bone.

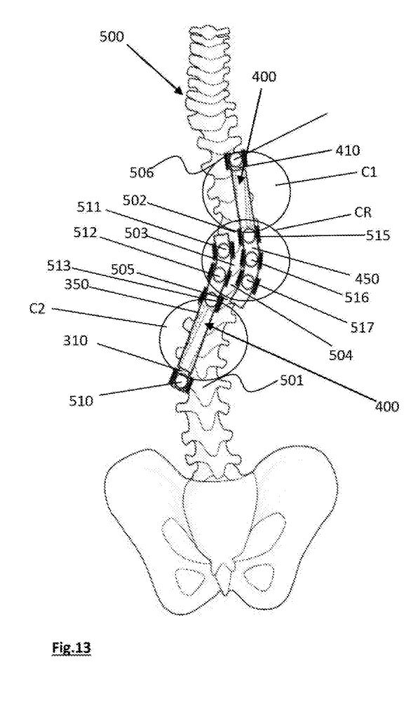

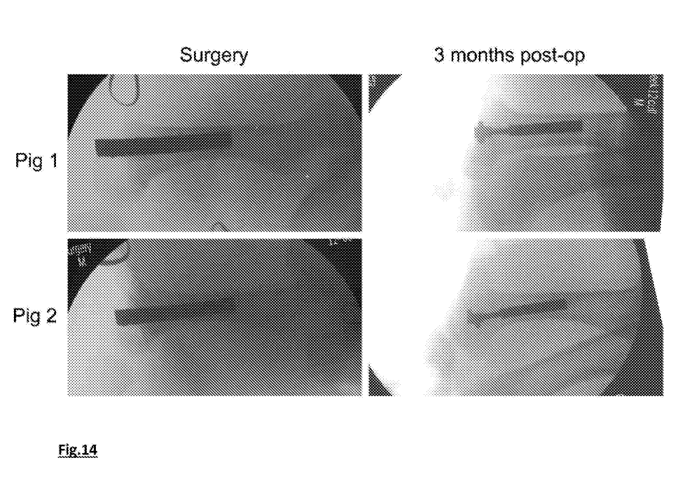

FIG. 13 shows an arrangement of orthopaedic devices 300 and 400 to correct a rotational deformed spine 500. As can be seen, the spine 500 is deformed and has a double curve. The central region (CR) of the spine has been fused. On each convexity of spine 500 (convexity C1 and convexity C2) a device 300 or 400 is positioned such that with future growth the spine will de-rotate, at least partially correcting the curvatures.

Particularly, orthopaedic devices 300 includes a first member 310 and device 400 includes a first member 410. Each device 300, 400 includes and a second member 350, 450 respectively. Second member 350 is configured to receive first member 310 and second member 450 is configured to receive first member 410.

In the example depicted in FIG. 13, first member 310 is secured to vertebra 501 by pedicle screw 510. Second member 350 is secured to vertebrae 502, 503 and 505 by respective pedicle screws 511, 512 and 513.

First member 410 is secured to vertebra 506 by pedicle screw 514. Second member 450 is secured to vertebrae 502, 503 and 504 by respective pedicle screws 515, 516 and 517.

As the subject grows, the first member 310 moves linearly and rotatably relative to second member 350 and first member 410 and second member 450 similarly moves linearly and rotatably relative to each other to correct the abnormal curvature of the spine 500 in regions C1 and C2.

### Experiment 1

Devices in accordance with the examples described above in relation to FIGS. 9a, 9b, 10a, 10b and 10c were manufactured in stainless steel GP1 using SLS methods, partially polished and sterilised by autoclave prior to implantation.

Surgery was conducted in two Large White/Landrace cross male piglets 10-15 kg (6-8 weeks old) (Pig 1 and Pig 2). Upon arrival, the animals were allowed two weeks to acclimatise prior to surgery.

The animals were sedated with 4.4 mg/kg Zoletil, 0.05 mg/kg Atropine and 2.2 mg/kg Xylazine. The operative site was shaved and wiped with iodine solution. Following intubation, isoflurane gas was delivered through inhalation during surgery to maintain sedation. The distal femur was accessed by a medial parapatellar approach, a 1.6 mm K-wire was used to find the centre of the femoral canal. The K-wire was then overdrilled with a 9 mm cannulated drill to fit the device. The device was inserted into the femoral canal and locked distally and then proximally using 4.5 mm HA coated half-pins under Image Intensifier guidance.

Post-surgery, animals were allowed to recover on heating mats and monitored overnight. Pigs were given Enrofloxacin (Baytril) 50 mg/kg IV for recovery and infection prevention for 6 days following surgery and 0.05 mg/kg Buprenorphine analgesic relief 2-3 times daily for 4 days following surgery. After the first post-op week, animals were checked twice a week and X-rayed monthly for three months. Animals were euthanised with Pentobarbitone 150 mg/kg IV after Zoletil 4.4 mg/kg IM sedation.

### Results

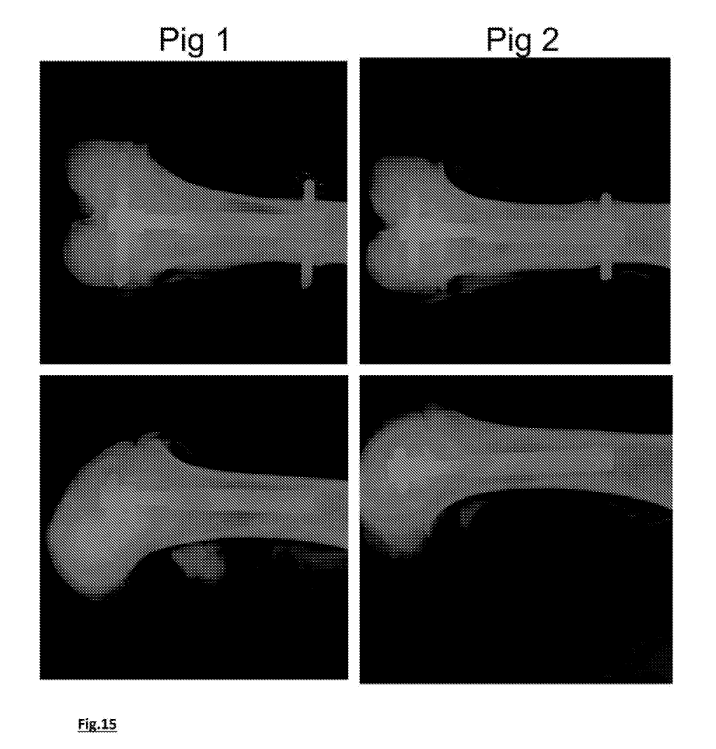

Longitudinal X-ray (FIG. 14) reveals that the devices lengthened in the three months between surgical implantation and harvest. X-rays of the implants ex vivo reveal an extension of 12.2 mm for Pig 1 and 13.8 mm for Pig 2 (FIG. 15).

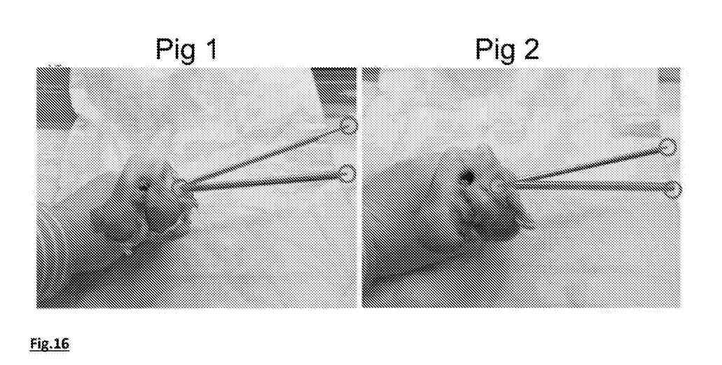

This lengthening correlated with a rotation of the device by 15° and 14° for Pig 1 and 2 respectively (see FIG. 16 and Table 1). CT scans compared to the non-operated side showed rotation differences of 8.4° and 11.2° for Pig 1 and 2 respectively. The differences between the device and bone rotation was likely due to slack in the device and/or bone remodelling.

TABLE 1
Measurement of the rotations of the
devices of Experiment 1 at the three
month harvest time from Pig 1 and Pig 2

| Measurement | Pig 1 | Pig 2 |
|---|---|---|
| Device rotation | 15° | 14° |
| Measured bone rotation | 8.4° | 11.2° |
| Difference between device | 5.6° | 3.8° |
| and bone rotation | | |

It will be appreciated by persons skilled in the art that numerous variations and/or modifications may be made to the above-described examples, without departing from the broad general scope of the present disclosure. The present examples are, therefore, to be considered in all respects as illustrative and not restrictive.

1. An orthopaedic device for securing between first and second regions of bone separated by a growth plate, the orthopaedic device comprising:
a first member having an end to secure to the first bone region; and a second member having an end to secure to the second bone region;
wherein the first and second members are moveably connected to each other such that when the ends are moved apart along a first axis, the first and/or second members rotate relative to each other about the first axis.

2. The orthopaedic device of claim 1, wherein the first and/or second members comprise one or more rotational feature.

3. The orthopaedic device of claim 2, wherein the first member extends from a first end to a second end and the first end is securable to the first bone region.

4. The orthopaedic device of claim 3, wherein the first member includes a first bone fixation region located at or adjacent to the first end of the first member and a mating region extending from said first bone fixation region.

5. The orthopaedic device of claim 4, wherein the second member extends from a first end to a second end, the first end securable to the second bone portion and the second end configured to receive at least part of the first member.

6. The orthopaedic device of claim 5, wherein the second member includes a second bone fixation region located at or adjacent to the first end of the second member.

7. The orthopaedic device of claim 4, wherein the second member includes a complementary mating region relative to the mating region of the first member.

8. The orthopaedic device of claim 4, wherein the one or more rotational feature of the first member is located on the mating region of the first member.

9. The orthopaedic device of claim 8, wherein the one or more rotational member is located on the complementary mating region of the second member.

10. The orthopaedic device of claim 2, wherein at least part of the second member has a lumen defined by an internal wall, said one or more rotational feature located on said internal wall.

11. The orthopaedic device of claim 10, wherein at least part of the first member is received in the lumen of the second member.

12. The orthopaedic device of any one of claim 11, wherein the one or more rotational feature of the first member comprises at least one ridge.

13. The orthopaedic device of claim 12, wherein the at least one ridge and/or at least one channel extend helically along a length of an outer surface of the first member.

14. The orthopaedic device of any one of any one claim 2, wherein said one or more rotational feature of the second member comprises at least one crest and/or at least one groove on the internal wall.

15. The orthopaedic device of claim 14, wherein the at least one crest and/or at least one groove extend helically along a length of the internal wall of the second member.

16. The orthopaedic device of claim 2, wherein the one or more rotational feature of the first member comprises at least one elongate surface.

17. The orthopaedic device of claim 16, wherein the at least one elongate surface extends helically along a length of the outer surface of the first member.

18. The orthopaedic device of claim 17, wherein the one or more rotational feature of the second member comprises at least one complementary guiding surface engageable with the at least one elongate surface.

19. The orthopaedic device of any claim 18, wherein the first member is substantially straight and the second member comprises at least a corresponding relatively straight terminal portion to receive the first portion of the first member therein.

20. The orthopaedic device of claim 19 wherein the second member includes one or more further portions which are angled relative to the terminal portion.

21. The orthopaedic device of claim 20, wherein the first member further includes a shoulder stop engageable with an end of the second member.

22-33. (canceled)
